(19) United States
(12) Patent Application Publication
(10) Pub. No.: US 2004/0039319 A1
(43) Pub. Date: Feb. 26, 2004

(54) Device and method for correcting knee genuflexus and equinus foot

(76) Inventor: Jesus Calatayud Carral, Zaragoza (ES)

Correspondence Address: WILLIAM COLLARD, COLLARD & ROE, P.C., 1077 NORTHERN BOULEVARD, ROSLYN, NY, 11576, US

(21) Appl. No.: 10/223,795

(22) Filed: Aug. 20, 2002

(51) Int. Cl. A61F 5/00

(52) U.S. Cl. 602/28

(57) ABSTRACT

A new method for correcting genu flexus and equinus foot, in order to achieve a normal step of the foot and the recurvatum of the knee, uses a device which consists of a pushing component and a traction component. The pushing component comprises a piece of suitable material in an appropriate form, which exerts a pressure on the supracondyle region of the femur. The position of the piece and the pressure it exerts is adjusted by means of a jack operated by a spindle, such that it is possible to adapt the device to different sizes and shapes of the legs of different patients. The traction component consists of a piece in the form of a receptacle that is attached to the ankle, the position and angle of which can be adjusted with respect to the axis of the leg. Once the traction component has been put in place, there are means for displacing it in the longitudinal direction, in the direction of the separation of the pushing component.

### BACKGROUND OF THE INVENTION

1. Field of the Invention

The present invention relates to a device for the correction of bending caused by genu flexus and equine foot, in order to achieve normality in the footstep and the recurvatum of the knee. The present invention also relates to a method for the correction of genu flexus and equine foot, in order to achieve normality in the footstep and in the recurvatum of the knee.

2. The Prior Art

Genu flexus is the permanent flexion of the knee, even though this flexion might be as small as an angle of 0. In other words, this is a deformity in the posterior concavity that is clearly observed in the lateral plane. It is as if the axes of the femur and the tibia in the sagittal plane formed a single straight line with no angle (angle 0, neutral line), but the articulation is unstable, and it can bend and fall, because the recurvatum or hyper-extension is not achieved. The genu flexus is the inability of the knee to reach hyper-extension or normal recurvatum.

Regarding the physiopathology, the antalgic posture of a few degrees of flexion maintained over time leads to capsular contractions and adherences that reach the posterior muscles. The PCL is shortened and, secondly, the action of the weight of the body centered towards the posterior overload of the tibial plates that finally give and collapse to form a neoarticulation with the posterior condyles. This process is stabilized with the production of posterior and anterior osteophytes and with the contact of the mass of the tibial spines and the intercondylar notch.

The knee, due to its high mobility, is able to support large forces from the powerful muscles that move the femur and tibia and also the additional force of the impact of inertia. Thus, the ligaments must stretch many times and almost always are accompanied by secondary traumatic arthritis. The quadriceps on the anterior face act as muscle forces and on the posterior face there are several well differentiated flexor muscles: the hamstrings (ischiotibial and popliteal muscles) and the superficial group of the calves (gastrocnemius, soleus and plantaris), the gastrocnemius being the most superficial, the largest and the most powerful of the entire calf group and the only one that flexes the leg.

Regarding pathological anatomy, the retractions and contractions involve the articular capsule, posterior cruciate ligament, popliteus and ischiotibial calf muscles, depending obviously on the severity of the signs and symptoms. Regarding bone defects, these are posterior, but depending on the association with other conditions, they may compromise one compartment more than another. Anterior and posterior osteophytosis may also be present.

There are different afflictions that can produce the signs and symptoms of genu flexus. These are signs and symptoms of inflammatory arthropathies: rheumatoid disease, reactive arthritis, arthritis and connective tissue disease, gout and crystal arthropathy; ligament lesions (medial ligament or external ligament), meniscus lesions with or without implication of the coronary ligament; internal displacements by meniscus or loss bodies; and lesions of the extensor mechanism.

All these afflictions can lead to the signs and symptoms of genu flexus, but the origin of the condition is apparent. The genu flexus can produce vertebral overload, gonarthrosis through overload of the cartilage through not being able to achieve normal recurvatum by which chondral wear, the formation of flat feet and hallux valgus are avoided. This is hidden and only the effects that it produces are apparent. Specific tests must be performed in order to be able to detect it.

All this leads to the suspicion that genu flexus is produced by a cause not apparent at first sight, such as a congenital malformation such as clubfoot, just as equinus foot has signs and symptoms that lead to lack of equilibrium of the muscles due to the power of the muscles of the calf, which, on growing, lead to an increment that is unnoticed in the first few months and years until the effects described above on the column, foot and the knee itself become manifest.

According to the static repair by Putti in the paralysis of the entire leg, the center of gravity of the upper part of the body should be moved backwards from the axis of the hips and immediately in front of the neutral line of the knee to obtain the full security mentioned, which will be 10° of hyper-extension. If this is not achieved even reaching the neutral line, bending of the knee is apparent, as shown in FIGS. 1 and 2. In the normal knee, the subject obtains the orthostatic state on attaining the aforementioned recurvatum, keeping on his or her feet without any effort, using only the weight of his or her body, due to the tension in the cruciate ligaments along with the counter-support given by the small elevation of the anterior side to the tibial articular surface on coming up against the femoral condyles, at the same time as inversion of the articulation is avoided. This means that the quadriceps, the large and only extensor muscle, is completely rested to act quickly and opportunely in the appropriate moment.

If there were no recurvatum, such as in the case of genu flexus or angle 0, the quadriceps would be exhausted from fighting against the orthostatic state with a wide range of deforming maneuvers to reduce the distressing orthostatic position, given that the aim of the quadriceps is not to maintain this position but rather to reach the recurvatum to achieve the rested orthostatic state.

There is a series of pathologies such as capsular lesion (greater limitation of the flexion than extension but always bent because of inflammatory rheumatoid arthropathy (sero-negative arthritis and connective disease), gout and crystal arthropathy), osteoarthritis (visible on an x-ray when it is clinically advanced, Bacquer cyst, hemarthrosis), traumatic arthritis (produced by tearing of the meniscus with or without implication of the corresponding coronary ligament, twisting of the medial collateral ligament, sprain of the cruciate ligaments, internal displacement of the meniscus or lost bodies, lesions of the extensor mechanism where quadriceps lesions are implicated, chondral malacia of the patella, patella-femoral arthrosis, relapsing dislocation of the patella), which occur in the knee itself, but we suspect that there is a common and obscure cause of the genu flexus that is hard to appreciate at the beginning, such as a congenital malformation, i.e., a variety of clubfoot such as equinus foot, produced by a muscular imbalance due to the strength of the muscles of the calf and subsequent failure in the coordination of these muscles.

Similarly, there is a series of pathogenic effects of genu flexus related with growth, which occur because they generate tension responsible for afflictions that appear in the bones of the articulations that are growing in the lower limb and even the vertebral column, above all, where there is less resistance, as in the parts of the bone in growth. Thus, the following signs and symptoms are present:

1. Anterior apophysitis of the tibia or Osgood-Schlatter disease. Often bilateral. Occurs in children aged 12 to 16 years, although it may occur in children under 10 years and over 23 years. It occurs earlier in girls.

2. Köhler disease I or tarsal scaphoiditis. Osteomalacia of the scaphoids only in children aged 3 to 10 years, almost always unilateral. Lesion due to strain, although it is a late-developing tarsus bone but it is more exposed because it is the key bone of the hollow that forms the internal arch of the foot, on suffering excess pressure through tension of the Achilles tendon in the equinus.

3. Köhler disease II or Freiberg disease. This is osteochondritis of the 2^{nd} metatarsal bone with malacia or epiphysary necrosis. It presents during adolescence and in adults. Coinciding with flat foot or in a fan, that is with disappearance of the transversal arch of the foot formed by the heads of the two metatarsal bones on supporting the load at the 2^{nd}, 3^{rd} and 4^{th} metatarsal bones.

4. Séller disease or posterior calcaneal apophysitis due to malacia where the Achilles tendon joins, which acts with great force on the muscles of the calves. It occurs in children of 3 to 10 years old and places the foot in equinus initiating another deformity just as common at this age, that of flat feet. If this is not corrected by eliminating the excess load from the calf muscles, in other words, the genu flexus, it will be a lifetime condition.

The basis of genu flexus is the permanent flexion of the knee, which supposes a great loss in stability, and leads to frequent falls in elderly people and subsequent hip fractures, thus compensatory positions are instinctively adopted to remedy this situation.

The patient leans forward by the lumbosacral joint to carry the centre of gravity of the body in front of the neutral line of the knees. This is the cause of back pain or lumbago due to the disk protrusions towards the posterior part of the vertebral column, causing sciatica. If the protrusion is facing forwards, there is the risk of osteochondritis in adolescents, juvenile kyphosis or Scheuermann disease or vertebral epiphysitis. In adults, the mushroom phenomenon occurs. Over time, these alterations cause the deterioration of the column in adults.

In the knee, on forcing the flexor imbalance, relief is sought from the flexus using the medial border for support leading to genu valgus. Other times, on the external border and genu varum is formed. The other harmful action of chronic genu flexus is the evolution towards gonarthrosis through exhausting the articular cartilage on being unable to reach recurvatum, the position that completely avoids overload of the articular cartilage and of the cartilage of the patella and femoral trochlea in a very special way.

In the foot, the Achilles tendon transmits the force of the short plantar flexor muscles of the calf through the insertion into the posterior apophysis of the calcaneum, placing the ankle in tension in equinus. If the body weight forces the calcaneum in valgus, it produces the inversion of the astragalus by rotating downwards and inwards, deteriorating the talus-calcaneum articulations, medio-tarsus and tarsus-metatarsus leading to the formation of flat feet and hallux valgus. Because of this force, alterations are produced in fibrous formations such as the deltoid ligament through chronic stretching secondary to the valgus foot, the talus-calcaneum ligaments and the Y ligament through talus-calcaneum articular distortions transversal to the tarsus and the tarsus-metatarsus. These alterations of fibrous formations listed above are produced by chronic stretching because of prolonged exposure to an orthostatic state or in patients with cavus feet (pes plantaris); but always through predominance of the short plantar flexor muscles of the calf as these act as limiters of dorsal flexion around 90°. This is where the denomination of short plantar flexors of the calf comes from.

This imbalance, which increases with age, is the cause of aches and pains through the metatarsalgia that it produces. Flat feet that begin early in infancy are likely to continue throughout the subject's life, unless action is taken to deal with the imbalance produced by the calf muscles.

Until the present, no physiotherapeutic or mechanic methods are known for correcting genu flexus and/or equinus foot, but rather, treatments are applied to the associated pathologies, such as, for example:

1.—LUMBAGO: rest on a hard bed, short waves, analgesic and myorelaxant medication and epidural anaesthesia infiltrations through the sacralis.

2.—SCIATICA: The same treatment as for lumbago and in some cases, removal of the disk.

3.—OSTEOCHONDRITIS, EPIPHYSITIS OR KYPHOSIS: with braces or specific exercises for that end.

4.—MUSHROOM: surgical treatment

5.—OSTEOARTHROSIS or GONARTHROSIS: with anti-inflammatories, bed rest, intra-articular filtrations of non-steroid anestesics.

6.—CHONDROMALACIA of the patella and the cartilage of the trochlea or femoral channel along which the patella slides: bed rest. If it is the result of relapsing dislocation of the patella it is treated surgically.

7.—FLAT FEET: with templates and specific exercises for treatment.

8.—PLANTAR FASCITIS: with padded troughs on the heel, corticoid infiltrations in the fascia plantar and even with surgery for removal of the calcaneal spur.

9.—HALLUS VALGUS: surgical intervention Some devices used for immobilising the leg have been described, for example in U.S. Pat. No. 4,407,277, which relates to a device that immobilises the leg to perform an arthroscopy. This is a static device that therefore does not cause any elongation during its operation.

### SUMMARY OF THE INVENTION

The present invention comprises a new method for correcting genu flexus and equinus foot, in order to achieve a normal step of the foot and the recurvatum of the knee, through the use of a device which consists of a pushing component and a traction component. The pushing component comprises a piece of suitable material in an appropriate form, which exerts a pressure on the supracondyle region of the femur. The position of the piece and the pressure it exerts is adjusted by means of a jack operated by a spindle, such that it is possible to adapt the device to different sizes and shapes of the legs of different patients. The traction component consists of a piece in the form of a receptacle that is attached to the ankle, the position and angle of which can be adjusted with respect to the axis of the leg. Once the traction component has been put in place, there are means for displacing it in the longitudinal direction, in the direction of the separation of the pushing component.

Optionally, the traction component has a plaster-carrier pedal, in order to increase the efficiency of the machine. With use of plaster, a more direct effect on the insertions of the gastrocnemius is achieved, as the foot is kept more firmly in the dorsal flexion position with the longitudinal arch of the foot fully corrected, thus avoiding the natural adjustments to prevent that force corresponding to the insertion from being exerted.

The piece that supports the plaster has the form of the foot, that keeps the position of the plaster firm without allowing it to move to the sides either longitudinally, as the pedal is fixed by two bolts or screws to two orifices that are drilled in the traction component, from which two tensing chains set the position of the plaster preventing any displacement.

The device is used for the correction of genu flexus and equinus foot, in order to achieve normality in the footstep and the recurvatum of the knee. The device is applied in those patients who have an excess force of the gastrocnemius, which is responsible for imbalance of the knee (genu flexus) and the foot (equinus foot). The gastrocnemius pulls from the heel and causes flexion of the knee producing, at the same time, equinus foot. On causing genu flexus, an angle of less than 180° occurs and therefore all the aforementioned clinical signs and symptoms occur.

In order to correct this motor imbalance, the device is applied by means of a shearing motion. In other words, the device is applied by compression and stretching at the point of insertion of the gastrocnemius on the posterior face of the inferior epiphysis of the femur (femoral condyles, zone adjacent to the posterior face of the inferior epiphysis of the femur and the posterior articular capsule of the knee). This produces a lengthening of these insertions to normalize the equilibrium of the muscles of the calf (short flexors of the calf) which are responsible for the genu flexus and equinus foot, as well as all the malformations that are produced in the foot. With this, it is possible to achieve a correction of flat foot and all the phases prior to this condition such as plantar fascitis, pain in the medio-tarsal, subastragaline and metatarsotarsal articulation. Similarly, clinical signs and symptoms related with lumbalgias, disk lesions, etc, are corrected. On the other hand, it prevents all those signs and symptoms related with lumbar overloads, hip fractures, the mushroom phenomenon in the columns of elderly people and osteochondritis in columns of young people.

With the present invention, it is possible to normalise the equinus in the foot with the sole of the foot resting normally on the ground. Normal recurvatum is achieved in the knee and the normal position of the body and vertebral disks is achieved in the column, with overload disappearing and the lumbar lordosis being regained.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 represents the different situations of extension-flexion around the neutral line;

FIG. 2 represents the situation of the center of gravity of the upper part of the body with respect to the neutral line of the knee according to the static reform in paralysis of the entire leg of Putti;

FIG. 3 represents a view in elevation of the device according to the invention;

FIG. 4 represents a plan detail of the traction component according to the invention;

FIG. 5 represents a side view of the device; and

FIG. 6 represents a detailed view of the plaster-carrier pedal according to the invention.

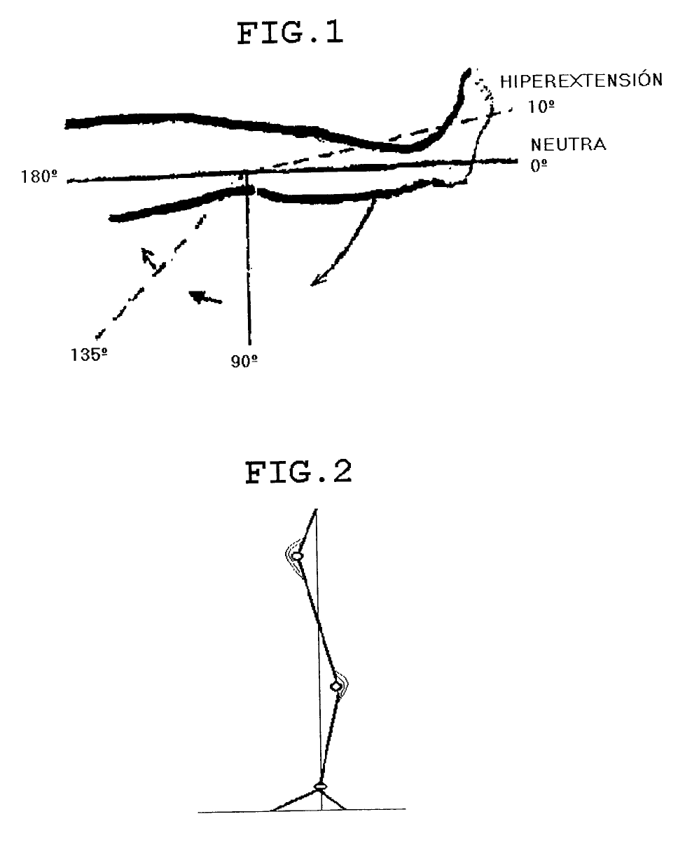

### DETAILED DESCRIPTION OF THE PREFERRED EMBODIMENT

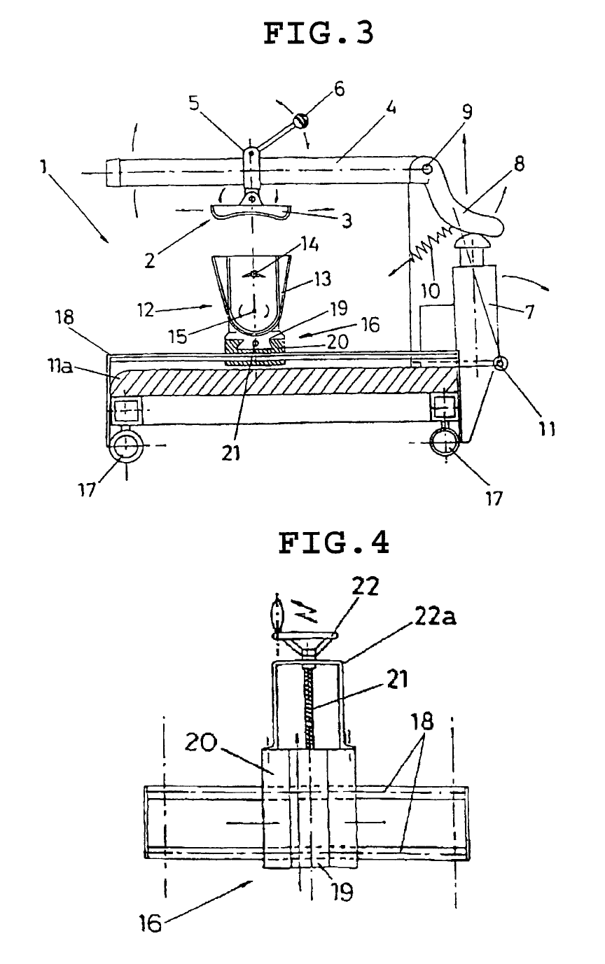

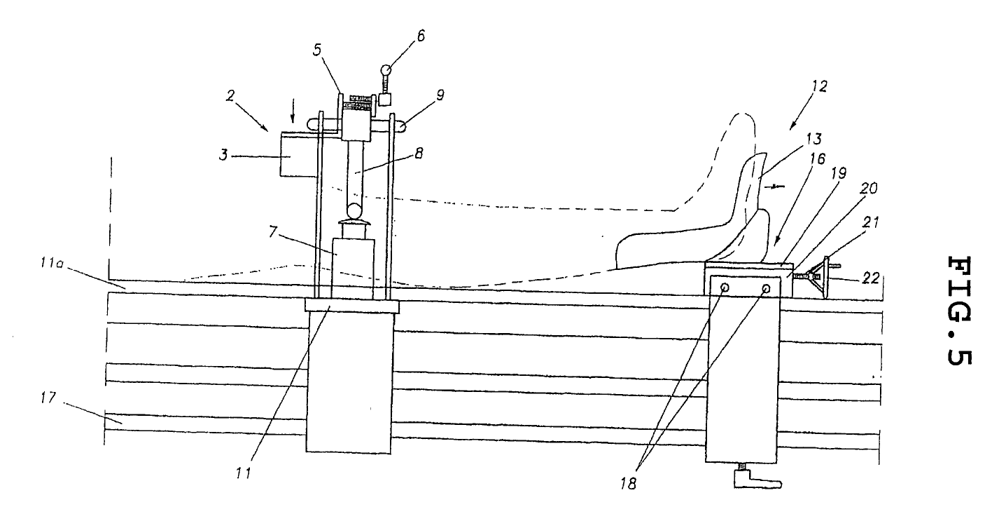

Referring now in detail to the drawings, FIGS. 3-5 show a machine 1 comprising a pushing component 2 consisting of a curved concave padded piece 3, mounted over a transversal crossbar 4, along which padded piece 3 can be displaced, and can be set in a given position by means of an eccentric 5 operated by a lever 6.

By means of a jack 7, a prolongation 8 of crossbar 4 is operated, which rotates about an axis 9, exerting pressure on the knee or releasing pressure, with spring 10 cooperating in the release of pressure.

The crossbar-spring-jack 4, 10, 7 hinges about an axis 11 to facilitate placing of the patient, who lies down over table 11a.

The traction component 12 consists of a support 13 that traps the foot, providing rotation about an axis 15 limited by a stop 14, to allow different foot angles to be adapted with respect to the axis of the leg.

Support 13 is mounted over a longitudinally and transversally displaceable structure 16 with respect to the leg, by means of pairs of perpendicular guides 17 and 18, which allows its adaptation to different shapes and lengths of legs, with the possibility of blocking the longitudinal movement.

Structure 16 comprises a piece 19 joined to support 13, which slides over another piece 20 which, in turn slides over guides 18, the coupling between pieces 19 and 20 consisting of a dovetail that only allows longitudinal displacement of one piece over another.

As shown in FIG. 4, this displacement is produced by a spindle 21 that screws over piece 19, and which is operated by a lever 22 that is attached to piece 20 by means of a support base 22a.

In this fashion, fixing the longitudinal position of the support and operating the lever, the leg will be stretched and the articulation fully extended until reaching the recurvatum, which in combination with the pressure exerted by the pushing component, manages to correct the genu flexus and/or equinus foot.

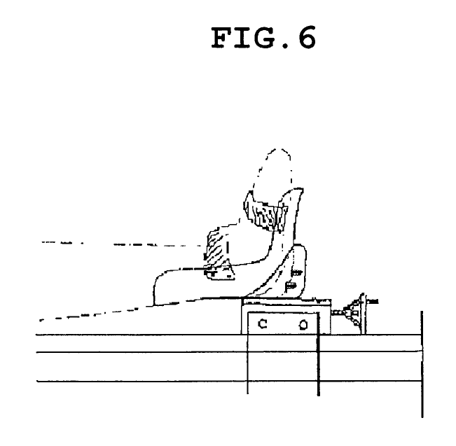

FIG. 6 represents a detailed view of the plaster-carrier pedal according to the invention. The piece that supports the plaster has the form of the foot, that keeps the position of the plaster firm without allowing it to move to the sides either longitudinally, as the pedal is fixed by two bolts or screws to two orifices that are drilled in the traction component, from which two tensing chains set the position of the plaster preventing any displacement.

Accordingly, while only a few embodiments of the present invention have been shown and described, it is obvious that many changes and modifications may be made thereunto without departing from the spirit and scope of the invention.

What is claimed is:

1. A method for the correction of genu flexus and equinus foot, in order to achieve normality in footstep and recurvatum of a knee of a patient, comprising:
applying a device that comprises a pushing component and a traction component, wherein the pushing component comprises a piece of suitable material that exerts pressure over a supracondyle region of a femur and the traction component comprises a receptacle that traps a heel; said step of applying comprising compressing and stretching a point of insertion of a gastrocnemius in a posterior face of an inferior epiphysis of a femur of the patient.

2. A method according to claim 1, wherein the pushing component is adjustable in terms of both position of the pushing component and pressure exerted by the pushing component, by means of a jack operated by a spindle.

3. A method according to claim 1, wherein the traction component is adjustable both in terms of position and angle with respect to an axis of a leg of the patient, and wherein the traction component has means for longitudinally displacing the traction component, in a direction of separation of the pushing component, after the traction component has been put in place.

4. A method according to claim 1, wherein the traction component has a plaster-carrier pedal to achieve a direct effect on the gastrocnemius insertions so that the foot is maintained firmly in a position of dorsal flexion with a longitudinal arch of the foot totally corrected, thus avoiding natural attempts to prevent force corresponding to the insertion from being applied.

5. A device for the correction of genu flexus and equinus foot, in order to achieve normality in footstep and recurvatum of a knee of a patient, comprising:
a pushing component comprising a piece of suitable material that exerts pressure on a supracondyle region of a femur of the patient; and
a traction component comprising a receptacle that traps the heel,
wherein the device is applied by means of shearing movement via compression and stretching of the point of insertion of the gastrocnemius in the posterior face of the inferior epiphysis of the femur.

6. Device according to claim 5, further comprising a jack operated by a spindle connected to the pushing component for adjusting the position and pressure exertion of the pushing component.

7. Device according to claim 5, wherein the traction component is adjustable in terms of position and angle with respect to the axis of the leg, and further comprising means for displacing the traction component in the direction of separation of the pushing component, after the traction component has been put in place.

8. Device according to claim 5, wherein the traction component has a plaster-carrier pedal to achieve a direct effect on gastrocnemius insertions, so that a foot of the patient is kept firmly in a position of dorsal flexion with a longitudinal arch of the foot totally corrected, thus avoiding natural attempts to prevent force corresponding to the insertion from being applied.
